(19) United States
(12) Plant Patent Application Publication
(10) Pub. No.: US 2006/0265796 P1
(43) Pub. Date: Nov. 23, 2006

(54) Peach tree named "FLATPRINCESSE"

(75) Inventors: Laurence Maillard, Elne (FR); Arsene Maillard, Elne (FR)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignee: S.A.R.L. AGRO SELECTION FRUITS, Elne (FR)

(21) Appl. No.: 11/434,798

(22) Filed: May 17, 2006

(30) Foreign Application Priority Data
May 18, 2005 (FR) 2005-0903

(51) Int. Cl. A01H 5/00

(52) U.S. Cl. PLT/195

(57) ABSTRACT

A new and distinct variety of flat peach tree, denominated ‘FLATPRINCESSE’, has a very high regular productivity and has the same good fruit quality as other varieties: ‘MAILLARFLAT’ ‘FLATPRETTY’ FLATQUEEN’ ‘FLATNICE’.

This application claims priority of Community plant variety right No. 2005/0903 filed on May 18, 2005 which is hereby incorporated by reference in its entirety.

Botanical classification: Prunus persica (L.) Batsch—White Flat peach.

### BACKGROUND OF THE VARIETY

The present invention relates to a new and distinct variety of prunus persica (L.) Batsch white flat peach tree. This tree, named ‘FLATPRINCESSE’, produces very long lifetime, good eating quality, semi-freestone flesh fruit for fresh market in July in the 66—Pyrénées Orientales—France. Contrast is made to ‘MAILLARFLAT’ flat peach, standard varieties, for reliable description. ‘FLATPRINCESSE’ is a promising candidate for commercial success in that it has a very high regular productivity and has the same well fruit quality as other varieties: ‘MAILLARFLAT’ ‘FLATPRETTY’ FLATQUEEN’ ‘FLATNICE’.

### ORIGIN OF THE VARIETY

‘FLATPRINCESSE’ flat peach tree (genotype) originated in a cultivated area of the south of France, in the 66—Pyrénées Orientales—France where it was tested. This place is under a Mediterranean climate in a temperate area characterized by irregular and low precipitation with drought in summer, high temperatures all year long. The ‘FLATPRINCESSE’ variety was obtained open-pollination of ‘MAILLARFLAT’. ‘FLATPRINCESSE’ was provisionally designated and tested as 02.03.21 PBPL and is registered at the Official Catalogue of the Agriculture Ministry of the French Republic Dec. 29, 2004 under number 1016569. It was obtained from open-pollination and is propagated by grafting. It has been determined to have unique tree and fruit characteristics making it worthy for commercial fresh fruit production. There are no known effects of this standard rootstock on this scion cultivar. Asexually propagated plants remained true to the original tree and all characteristics of the tree and the fruit were transmitted. The plant was reproduced asexually situated at Les Régalines, Route d'Alenya, La Prade de Mousseillous, 66200 ELNE, Pyrénées Orientales, France.

### SUMMARY OF THE VARIETY

The new and distinct variety of flat peach tree blooms with ‘FLATPRINCESSE’ flat peach in March at Perpignan in the Pyrénées Orientales, France. More particularly, it blooms at the same time as ‘MAILLARFLAT, between 7^{th} and 21^{st} of March.

The fruit of ‘FLATPRINCESSE’ first ripens in August 18 days after ‘MAILLARFLAT’. More particularly, it approximately ripens between August 16^{th} and 25^{th}.

### DESCRIPTION OF THE DRAWINGS

In the accompanying drawing, which are as nearly true as it is reasonably possible to make in a colour illustration of this type:

FIG. 1 is a colour photograph which shows the flesh of the fruit of the new variety ‘FLATPRINCESSE’.

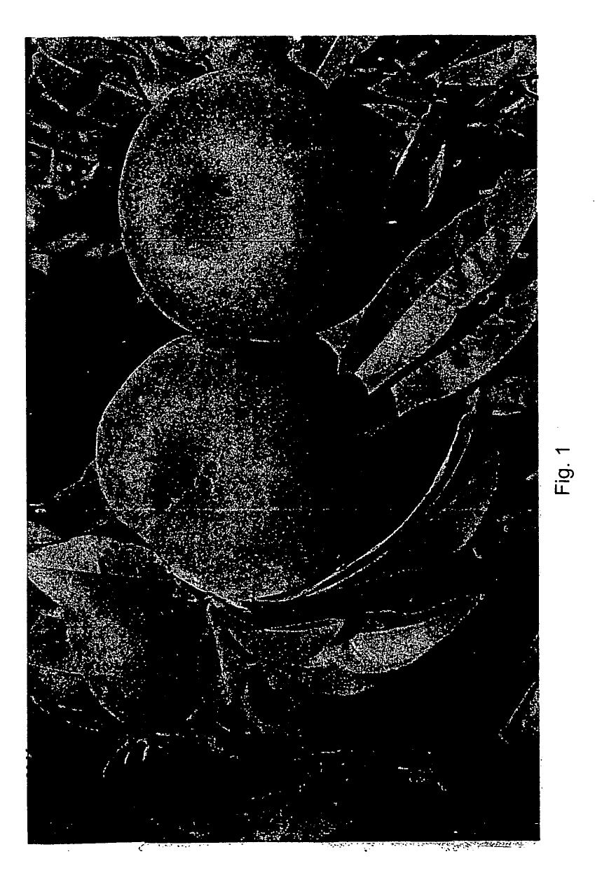

FIG. 2 is a colour photograph which shows a typical specimen of the fruit of the new variety ‘FLATPRINCESSE’.

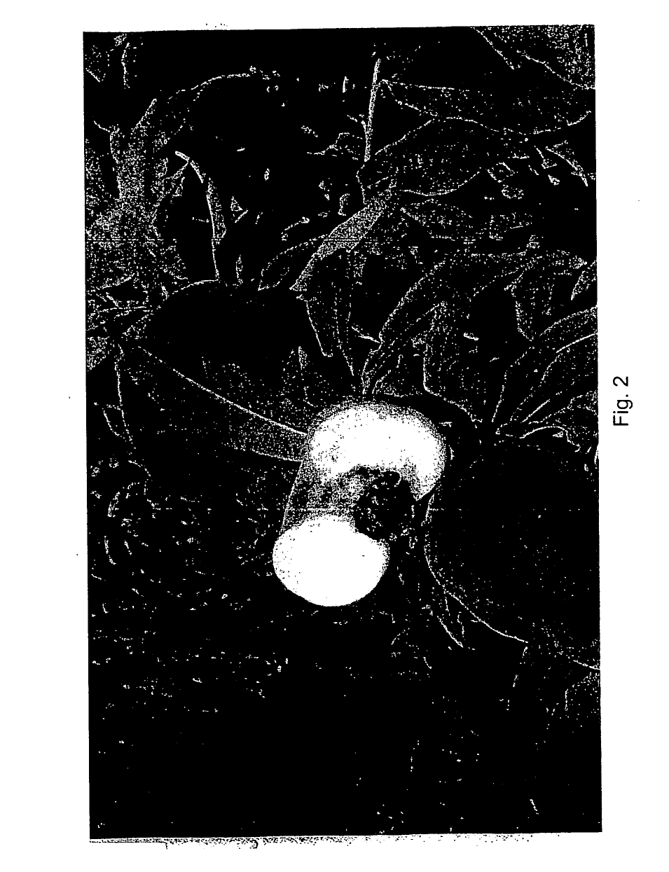

### DETAILED BOTANICAL DESCRIPTION

The tree, flowers, and fruit may vary in slight detail due to variations in soil type, cultural practices, and climatic condition. The potential for commercial production of fresh fruit by ‘FLATPRINCESSE’ is high, due to its early ripening, its evenness of maturity.

Trees are vigorous and medium stature half-standing in a semi-spread out aspect. The flowering shoot is present excluding brushwoods side away from sun. Flowering begins medium in springtime. The type of flower is showy and rosaceous with relative medium petal size. Petals are pink. Two or three flat peach leaf glands are present and reniform. Time of maturity for consumption is late. The fruit flesh is white and its skin is red. The stone is small size and the flesh is semi adherent.

Compared to ‘MAILLARFLAT’ variety, ‘FLATPRINCESSE’ has an older time of maturity, 18 days later. Fruits are red coloured over 90% instead of 60 to 70% for ‘MAILLARFLAT4 fruits. ‘FLATPRINCESSE’ has more flowers, 45 flowers per meter instead of 40 flowers per meter for ‘MAILLARFLAT’. ‘FLATPRINCESSE’ fruit tastes the same as ‘MAILLARFLAT’, semi sweet and aromatic. Evenness of productivity is better and regular.

### DETAILED DESCRIPTION

Referring more specifically to the pomological details of this new and distinct variety of flat peach tree, the following has been observed during the fruiting season under the ecological conditions prevailing at Elne which is located in the Pyrénées Orientales, France.

### Tree

Size:
   Generally.—Considered medium and large as compared to other common commercial flat peach cultivars ripening in the middle season of maturity.
Vigor: Considered vigorous.
Productivity: Very Productive. Fruit set is spaced by thinning to develop the remaining fruit into the desired market sized fruit. The number of the fruit set varies with the prevailing climatic conditions, and cultural practices employed during the bloom period, and is therefore not distinctive of the present variety.
Bearer: Regular. Fruit set has been heavy during the years of observation and thinning was necessary during the past 3 years.
Form: Upright semi-standing, and pruned to a vase semi-spread out shape.
Density: Considered dense.
Hardiness: The present tree was grown and evaluated in France. The variety appears to be hardy under typical central Pyrénées Orientales climatic conditions.

### Trunk

Diameter: Considered average.
Bark texture: Considered moderately rough, with numerous folds of papery scarfskin being present.
Lenticels: Numerous flat, oval lenticels are present.
Lenticel colour: Considered brown.
Bark colouration: Variable, but it is generally considered to be a medium grey-brown.

### Branches

Size: Considered medium for the variety.
Diameter: Average as compared to other flat peach varieties.
Surface texture: Average, and appearing furrowed on wood which is several years old.
Crotch angles: Primary branches are considered variable. This particular characteristic is not considered distinctive of the variety, however.
Current season shoots:
   Surface texture.—Substantially glabrous.
Internode length: Considered average.
Colour of mature branches: Medium brown.
Current seasons shoots:
   Colour.—Light green. The colour of new shoot tips is considered a bright and shiny green.

### Leaves

Size: Considered medium to large for the species. Leaf measurements have been taken from vigorous, upright, current-season growth at approximately mid-shoot.
Leaf length: Considered average.
Leaf width: Considered average.
Leaf base shape: Slightly oblique relative to the leaf longitudinal axis.
Leaf form: Lanceolate. Considered to be relatively narrow for the species.
Leaf tip form: Reniform.
Leaf colour:
   Upper leaf surface.—green.
Leaf texture: Glabrous.
Leaf colour: green.
   Lower surface.—Medium green.
Leaf venation: Pinnately veined.
Mid-vein:
   Colour.—Light white green.
Leaf margins: Slightly undulating.
Form: Considered crenate.
Uniformity: Considered generally uniform.
Leaf glands:
   Size.—Considered medium.
   Length.—About 8.0 to about 10.0 mm.
   Diameter.—About 1.0 to about 2.0 mm.
   Colour.—Pale green.
Leaf glands:
   Size.—Considered medium.
   Number.—Generally one gland per margin side. Occasionally two glands per margin side.
   Type.—Reniform.
   Colour.—Considered a pale orange.
Leaf stipules:
   Size.—Medium large for the variety.
   Number.—Typically 2 per leaf bud and up to 4 per shoot tip.
   Form.—Lanceolate in form and having a serrated margin.
   Colour.—Green, when young, but graduating to a brown colour, with advancing senescence. The stipules are considered to be early deciduous.

### Flowers

Flower buds:
   Generally.—The floral buds, depending upon the stage of development, are conic in form; and slightly appressed relative to the bearing shoot.
Flower buds:
   Colour.—This characteristic is dependent upon the proximity to bloom. The bud scales are deep purple. The buds are considered hardy under typical central Pyrénées Orientales climatic conditions.
Hardiness: No winter injury has been noted during the last several years of evaluation in the central Pyrénées Orientales. The current variety has not been intentionally subjected to drought or heat stress, and therefore this information is not available.
Date of bloom: early March
Blooming time: Considered early-season in relative comparison to other commercial flat peach cultivars grown in the Pyrénées Orientales, France. The date of full bloom is observed on March. The date of bloom varies slightly with climatic conditions and cultural practices.
Duration of bloom: Approximately 15 days. This characteristic varies slightly with the prevailing climatic conditions.
Flower type: The variety is considered to have a showy rosaceous type flower.
Flower size: Flower diameter at full bloom approximately 34.0 to about 38.0 millimeters.
Bloom quantity: Considered very abundant, approximately 45 flowers per meter.
Flower bud frequency: Normally 1 to 2 flower buds appear per node.
Petal size:
   Generally.—Considered medium to large for the species.
Length: Considered average.
Width: Considered average.
Petal form: Slightly ovoid.
Petal count: Nearly always 5.
Petal texture: Glabrous.
Petal colour: Pink.
Fragrance: Slight.
Petal claw:
   Form.—The claw is considered generally ovoid and has a medium size when compared to other varieties.
   Length.—Considered average.
   Width.—Considered average.
Petal margins: Generally considered variable, from nearly smooth to slightly ruffled, to moderately undulate.
Petal apex:
   Generally.—The petal apices are generally entire at the tip.
Flower pedicel:
   Length.—Considered medium-long.
   Diameter.—Considered average.
   Colour.—A medium brown.
Floral nectaries:
   Colour.—A dull orange.
Calyx:
   Surface texture.—Generally glabrous.
   Colour.—Purple.
Sepals:
   Surface texture.—The surface has a short, fine pubescent texture.
   Size.—Average, and ovate in form.
   Colour.—A deep purple.
Anthers:
   Generally.—Average in length.
   Colour.—Red to reddish-orange dorsally.
Pollen production: Pollen is abundant, and has a white colour. The present variety is considered self fruitful (self-pollinating).
Filaments:
   Size.—Length is variable and is considered average.
Colour: Considered light pink.
Pistil:
   Number.—Usually 1, rarely 2.
   Generally.—Average in size.
   Length.—Considered average. including the ovary.
   Colour.—Considered a very pale green. Surface texture.—The variety has a long glabrous pistil.

### Fruit

Maturity when described: Firm ripe condition (shipping ripe).
Date of picking: August. The date of harvest varies slightly with the prevailing climatic conditions.
Size:
   Generally.—Considered large, and uniform.
Average cheek diameter: Considered average.
Average axial diameter: Considered average.
Typical weight: Approximately between 150.0 and 200.0 grams. This characteristic is high dependent upon the prevailing cultural practices, and therefore is not particularly distinctive of the variety.
Fruit form:
   Generally.—Considerably oblate and flattened. The fruit is generally uniform in symmetry.
Fruit suture: Very shallow and smooth, extending from the base to the apex. No apparent callousing or stitching exists along the suture line.
Suture:
   Colour.—This has a pale brown background colour.
Ventral surface:
   Form.—Only slightly indented.
Apex: Rounded.
Base: Generally retuse.
Stem cavity: Generally elongated in the suture plane.
Fruit skin:
   Thickness.—Considered very thick.
   Texture.—Low Pubescent.
   Taste.—semi sweet, very aromatic.
   Tendency to crack.—None observed.
Colour:
   Blush colour.—This blush colour is generally red. The red blush covers 80 to 90% of the fruit skin surface. The percentage of the blush on the fruit skin surface can vary, and is generally dependant upon the prevailing conditions under which the fruit was grown.
   Ground colour.—Pale brown.
Fruit stem: Medium in length.
Diameter: Considered average.
Colour: Pale white-green.
Flesh:
   Ripens.—Evenly.
   Texture.—Very firm, juicy and dense.
   Fibers.—Few, small, and tender ones are typically found.
   Aroma.—Very slight.
   Eating quality.—Considered very good.
   Flavor.—Considered semi sweet and sub acidic. The flavor is considered both pleasant and balanced.
   Juice.—Moderate.
   Brix.—This characteristic varies slightly with the number of fruit per tree; prevailing cultural practices; and the surrounding climatic conditions.
   Flesh colour.—White.

### Stone

Type: Clingstone.
Size: Considered medium for the variety. The stone size varies significantly depending upon the tree vigor, crop load and prevailing growing conditions.
Length: Average.
Width: Average.
Diameter: Average.
Form: Ovoid.
Base: The stone is usually rounded to slightly oblique relative to the ventral side.
Apex:
   Shape.—The stone apex has a small prominent tip.
Stone surface:
   Surface texture.—The pit is irregularly furrowed toward the apical end. Pitting is generally more present and noted on the lateral sides and toward the base.
   Ridges.—The surface texture is generally characterized by more prominent ridges along the ventral edges and is more prominent at the apical tip.
Ventral edge:
   Width.—Considered medium.
Dorsal edge:
   Shape.—Full, lightly grooved, and having a reasonably smooth margin.
Stone colour: The colour of the dry stone is generally considered a reddish brown.
Tendency to split: Splitting has rarely been noted.
Kernel:
   Size.—The kernel is considered medium-large.
   Form.—Considered ovoid.
   Pellicle.—No pubescent.
   Colour.—brown-orange.
Use: The subject variety ‘FLATPRINCESSE’ is considered to be a flat peach tree of the medium season of maturity, and which produces fruit that are considered firm, attractively coloured, and which are useful for both local and very long distance shipping.
Keeping quality: Excellent. Fruit stayed one week on tree before harvest and then, has stored well up to 3 weeks after harvest at 1.0 degree Celsius.
Shipping quality: Considered good. The fruit of the new flat peach variety showed minimal bruising of the flesh or skin damage after being subjected to normal harvesting and packing procedures.
Resistance to insects and disease: No particular susceptibilities were noted. The present variety has not been tested to expose or detect any susceptibilities or resistances to any known plant and/or fruit diseases.

Although the new variety of flat peach tree possesses the described characteristics when grown under the ecological conditions prevailing near ELNE, FRANCE, it should be understood that variations of the usual magnitude and characteristics incident to changes in growing conditions, fertilization, pruning, pest control and horticultural management are to be expected.

We claim:

1. A new and distinct flat peach tree variety as illustrated and described, characterized by a very high regular productivity and has the same good fruit quality as other varieties: ‘MAILLARFLAT’ ‘FLATPRETTY’ FLATQUEEN’ ‘FLATNICE’.
